(19) United States
(12) Patent Application Publication
(10) Pub. No.: US 2004/0121451 A1
(43) Pub. Date: Jun. 24, 2004

(54) Treatment of sols, gels and mixtures thereof

(76) Inventors: Niko Moritz, Turku (FI); Ilkka Kangasniemi, Piispanristi (FI); Antti Yli-Urpo, Littoinen (FI); Timo Peltola, Turku (FI); Mika Jokinen, Turku (FI)

Correspondence Address: James C Lydon, Suite 100, 100 Daingerfield Road, Alexandria, VA, 22314, US

(21) Appl. No.: 10/472,113

(22) PCT Filed: Mar. 15, 2002
(86) PCT No.: PCT/FI02/00209

(30) Foreign Application Priority Data
Mar. 16, 2001 (FI) 20010523

(51) Int. Cl. C12M 1/34; B05D 3/00

(52) U.S. Cl. 435/287.2; 427/2.11

(57) ABSTRACT

A method for at least partially treating biologically active sols, gels, mixtures or composites of sols and gels, and/or sol-gel derived materials, the sols, gels, mixtures or composites of sols and gels, and/or sol-gel derived materials having OH-groups and being at least partially amorphous, the treatment being performed with a localised electromagnetic and/or acoustic energy. A method for coating a device with biologically active sols, gels, mixtures or composites of sols and gels, and/or sol-gel derived materials and a method for modifying the biological activity of biologically active sols, gels, mixtures or composites of sols and gels, and/or sol-gel derived materials are also disclosed. Both methods include treatment with localised electromagnetic and/or acoustic energy. Also disclosed is the use of the method to fabricate different devices and to attach at least two devices.

The object of the invention is to provide a method for at least partially treating biologically active sols, gels, mixtures or composites of sols and gels, and/or sol-gel derived materials, a method for coating a device with biologically active sots, gels, mixtures or composites of sols and gels, and/or sol-gel derived materials and a method for modifying the biological activity of biologically active sols, gels, mixtures or composites of sots and gels and/or sol-gel derived materials.

### BACKGROUND OF THE INVENTION

Silica and Titania Gels

Silica-xerogels (xerogel=dried gel) are partially hydrolysed oxides of silicon. Hydrolysed oxide gels can be produced by a sol-gel process, which has been used for producing ceramic and glass materials for many years. Most commonly, the process for producing sols is based on hydrolysis of a metal-alkoxide and subsequent polymerisation of the metal hydroxides as follows:

1) Si(OR)₄+H₂O→HO—Si(OR)₃+ROH

2) HO—Si(OR)₃+3H₂O+ROH→Si(OH)₄+4ROH

3) Si(OH)₄+Si(OH)₄→(HO)₃Si—O—Si(OH)₃+H₂O

The sol may also contain other additives such as acids or bases used for catalysing the reaction. When the sol obtained is further polymerized, the polymerisation reaction goes further, and additional chains, rings and three-dimensional networks and particles are formed, and a gel comprising water, the alcohol of the alkoxy group is formed. If alcohol and water are then extracted from the gel by washing and evaporating, a xerogel is obtained.

During drying large shrinking occur creating internal stresses into the gel. If the monolithic gel is not allowed sufficiently time to relax its internal stresses, it will crack. If the gel is produced in small dimensions as particles, fibres or coatings these stresses can be avoided almost completely.

During drying further polymerisation of the remaining OH-groups also continues. The continuing polymerisation carries on for a rather long time after gelation. This is called ageing. The further the polymerisation goes on, the more stable the gel or xerogel becomes. However, at room temperature the polymerisation will in practice stop after a few weeks ageing and the xerogel will not become totally stable. If the temperature is raised, the polymerisation reaction can be accelerated, further stabilisation and shrinkage occurs, and more internal stresses are introduced into the xerogel.

If the temperature is raised high enough (around 1000° C. for monolithic SiO₂-gels) the gel or xerogel becomes a pure oxide and there are no OH-groups present in the material.

The silica-xerogel material has been found to be biocompatible. In other words, it does not adversely affect the surrounding tissue, e.g. by causing an inflammation reaction.

Corresponding systems are used also for titania gels and similar properties are reached. Sol-gel derived titaniagel (TiO₂) and titania-silica films and coatings are widely used, also in bioceramics. Pure titanium implants have been, among many other metals, used in implantology and orthopedics to replace damaged bone tissue. However, the contact with bone is relatively poor and it has been suggested that several clinical problems could be avoided if the tissues could better adhere to the surface of the implant. TiO₂ coatings on titanium implants have been shown to be a potential help for better bone bonding. A biomaterial consisting of a thin oxide layer on a mechanically tough titanium substrate provides a possibility to design devices that satisfy the dual requirement for clinical use, i.e., a stable interface with connective tissue and an appropriate, functional match of the mechanical behaviour.

In prior art, slow release of a drug for example has been referred to as longer release times than a few days. The longest release time varies for xerogels depending on how they are produced. In general however, all xerogels so far have had a maximum obtainable release time up to one year.

In several publications, for example in “Structural change in sol-gel derived SiO₂ films using ultraviolet irradiation”, Imai et al., SPIE, Vol, 2283, Sol-gel optics III, 1994, pages 71-76, it is mentioned that SiO₂—TiO₂ sol-gel materials strongly absorb light in the UV and IR spectral regions, thus light can be used to gelate and/or densify these materials. These materials are however not bioactive. On the other hand, various uses of electromagnetic radiation for processing sol-gel derived materials are known. Examples of such uses are

direct selective laser densification of silica-titania glass sol-gel films in the fabrication of optical waveguides,

direct selective laser densification of oxide silica-titania sol-gel films in the fabrication of optical gratings,

densification of silica monoliths,

densification of sol-gel silica and titania films by synchrotron and ultraviolet radiation,

microwave processing of silica and doped-silica glasses by sol-gel, and

photo-patterning of silica and titania films using ultraviolet radiation.

The patent publication U.S. Pat. No. 5,490,962 discloses as method for preparation of medical devices by solid free-form fabrication methods. One example of said method is selective laser sintering (SLS). The method consists of sintering a mixture of biocompatible polymer and a biologically active agent.

Mixing of Organic and Inorganic Molecules

The general principles of mixing organic substances with gels are well known. The basic idea is that an organic substance is added to the sol-stage of the sol-gel process. Then, after gelation, the organic part has become an inherent part of the material. In conventional manufacturing processes of corresponding materials, this is not possible because the temperatures are much too high for organic substances to survive.

The processing temperature is naturally a limiting factor for many substances in for example organically modified silicates (also called ORMOSILS). In the case of medicines, the temperature is limited by the breakdown of the structure or functionality of the medicine. For proteins, enzymes, antibodies and whole cells, the temperature limit is as low as 40° C. since they will start coagulating at and above that temperature.

Organic substances are generally added to silica gels to modify the natural properties of the silicates with those of the organic substances. Some combinations of dopants and matrices used thus far are disclosed in Chemistry of Materials (1994) 6:1605-1614 (D. Avnir et al.).

Delivery Techniques

Silica sol-gel material directed for oral short-term (less than 24 hours) drug delivery has been described in the prior art. For example Unger, K. et. al. describe methods of mixing drugs with silica-viscous sol in “Drug Development and Industrial Pharmacy” (1983) 9 (1&2) 69-91. The article describes drug dispersions as crystals, particles or liquid mixed with the partially polymerised viscous liquid, polyethoxysiloxane (PES), which was produced by partially hydrolysing tetraethoxysiloxane and then partially condensing it with an acidic catalyst. Further, the polycondensation was continued by adding a basic catalyst and water. The basic catalyst was not needed if the drug was basic. The drug dispersions were insoluble in the PES liquid, and the drugs could be encapsulated within silica-gel envelopes in this way as the gelling proceeded. It can be seen from the release profiles of the article that the drug release was based on the porosity of the gel.

Unger et al. also describe a method of polycondensation in solution, which starts with mixing PES with a solution of the drug in an appropriate solvent, giving a molecular scale entrapment of the drug in the polymer. The material obtained from polycondensation in solution is hard and brittle. The release rate of the drug is controlled by diffusion through the pores of the matrix material. The release rate curves for the molecularly dispersed materials are typical of those of diffusive release. The release of 100% of codeine at pH 2.2 lasted around 14 h in the in vitro experiment.

The xerogels described above are limited in following aspects: since they cannot be heat-treated at elevated temperatures, the ultimate degree of polycondensation is limited, it is not possible to make the materials totally inert and it is not possible to make coatings and bulk materials slowly degradable. Therefore an object of the invention is to introduce a way of further advancing the polycondensation, densification, consolidation or sintering without increasing the temperature. It is also an object of the invention to provide a method for producing medical devices with a controllable biological activity.

### BRIEF DESCRIPTION OF THE INVENTION

The invention is characterised by what is mentioned in the appended claims.

The invention relates to a method for at least partially treating biologically active sols, gels, mixtures or composites of sols and gels and/or sol-gel derived materials, said sols, gels, mixtures or composites of sols and gels, and/or sol-gel derived materials comprising OH-groups and being at least partially amorphous, the said treatment being performed with a localised electromagnetic and/or acoustic energy.

The invention also relates to a method for coating a device with biologically active sols, gels, mixtures or composites of sols and gels, and/or sol-gel derived materials. Said method is characterised in that at least one layer of biologically active sols, gels, mixtures or composites of sols and gels, and/or sol-gel derived materials is deposited on the surface of the device and that each said layer is treated with a localised electromagnetic and/or acoustic energy prior to the eventual deposition of another layer of biologically active sols, gels, mixtures or composites of sols and gels, and/or sol-gel derived materials.

The invention still relates to a method for modifying the biological activity of biologically active sols, gels, mixtures or composites of sols and gels, and/or sol-gel derived materials by treating at least portion of the surface of the biologically active sols, gels, mixtures or composites of sols and gels, and/or sol-gel derived materials with a localised electromagnetic and/or acoustic energy. The invention further relates to different devices prepared by the method of the invention, such as fibres, monoliths, granulates, woven or nonwoven mats, tissue-guiding devices, films and coatings.

### DETAILED DESCRIPTION OF THE INVENTION

The invention relates to method for at least partially treating biologically active sols, gels, mixtures or composites of sols and gels, and/or sol-gel derived materials, said sols, gels, mixtures or composites of sols and gels, and/or sol-gel derived materials comprising OH-groups and being at least partially amorphous, the said treatment being performed with a localised electromagnetic and/or acoustic energy.

According to an embodiment of the invention, said treatment consists of polycondensation, consolidation, densification and/or sintering.

Now it has been surprisingly found out that by treating sols, gels, mixtures or composites of sols and gels, and/or sol-gel derived materials with electromagnetic and/or acoustic energy it is possible to create a surface, which is able to enhance transfer of inorganic ions, such as calcium and phosphate ions, from the surrounding liquid to the surface. In practice this means, for example, that an implant surface coated with a gel or a sol-gel derived material and treated according to present invention is able to induce calcium and phosphate ions from body liquids when the implant is planted into a mammal. This enables fast and abundant formation of hydroxyapatite layer on the implant surface on treated areas after the implant is placed into a patient, so the biological patterning will occur when the surface comes into a contact with tissue. In conventional surface treatment methods it is necessary to cover or coat the implant with hydroxyapatite layer before placing it into a patient.

According to the present invention the sol, gel, mixtures or composites of sol and gel, and/or sol-gel derived materials can be locally treated with electromagnetic and/or acoustic energy so that they can enhance the fast and abundant formation of hydroxyapatite. Use of electromagnetic and/or acoustic energy enables patterning of the treated sol, gel, mixtures or composites of sol and gel, and/or sol-gel derived materials. The present invention makes this patterning easy and simple. For example, when using a laser for the treatment of a sol, gel, mixtures or composites of sol and gel, and/or sol-gel derived materials according to the present invention, it is very easy to choose and define the areas, which will be treated. In practice, for example the width of the laser beam can be used for defining the width of the treated areas.

The invention thus introduces a way of treating the biologically active sols, gels, mixtures or composites of sols and gels, and/or sol-gel derived materials without increasing the temperature and therefore it is possible to make coatings and bulk materials slowly degradable. According to an embodiment of the invention, the invention allows advancing the polycondensation, consolidation, densification or sintering of the material without using elevated temperature. Indeed, the temperature is elevated only moderately and on a restricted area of the material at a time. The invention thus allows the production of biocompatible ceramics or composites thereof for medical or technical applications at room temperature or at low temperatures using localised energy source as a catalyst for the treatment of ready gelated material (gel), dried gel (xerogel), sol-gel derived ceramic material or otherwise derived material, partly gelated material (mixture of sol and gel), a solution containing the ceramic ingredients (sol), or a mixture or a composite of one of the preceding materials with another component(s).

The invention also allows the production of materials having a controlled porosity in the nano-level, leading to materials that have poor optical properties. Furthermore, with the method according to the invention, it is possible to firstly fill the pores of a porous material with for example a sol and then treat this material in order to obtain a sort of composite material wherein the original porous material forms the matrix.

The sols, gels, mixtures or composites of sols and gels, and sol-gel derived materials that have been treated according the invention comprise OH-groups and are at least partially amorphous. Amount of amorphous fraction in treated material is typically relatively high. Number of OH-groups on the surface of the treated material may typically be 2-60H-groups/nm², i.e. the number of the surface of groups is higher than in materials treated by conventional methods. The number of OH-groups is preferably 2-40H-groups/nm². The number of OH-groups can be controlled by the choice of parameters during the process, e.g. by choosing the precursor/water ratio, temperature and the catalyst concentration so that a low condensation rate is obtained. Low concentration rate is able to provide a higher number of OH-groups on the material surface.

By biocompatibility it is meant the ability of a material to perform with an appropriate host response in a specific application. By bioactive material, it is meant a material that has been designed to induce specific biological activity and by biomaterial, it is meant a non-viable material used in a medical device, intended to interact with biological systems. Biocompatible and bioactive materials may be biodegradable or not.

A sol is a stable suspension, where the solid phase is uniformly distributed in a surrounding liquid phase. The solid phase has been formed in the same liquid phase, which usually contains water, through different chemical reactions, such as hydrolysis and/or condensation. The precursors, which are used for forming the sol, are organic solutions, such as alkoxides, or inorganic silicates. Preferably organic precursors are used.

Sol is transformed into a gel via further condensation reaction or reactions caused by ageing of the sol and/or by drying and/or by a heat treatment. In other words a gel is obtained by a chemical reaction or a thermal treatment of a corresponding sol.

A material is defined to be a gel when it is formed from the sol, which has been dried and/or heat-treated at ≦700° C.

Sol-gel derived materials are materials that are formed in a process, preferably in a continuing process, starting from organic or inorganic precursors, such as alkoxides or inorganic silicates. A sol formed through chemical reactions is being used to form a gel via further chemical reactions caused by ageing of the sol, such as condensation reaction. A sol-gel derived material can also be a dried powder, which has been prepared according to sol-gel technique before drying at temperature ≦700° C. Sol-gel derived materials comprise preferably TiO₂, SiO₂ or TiO₂.SiO₂ based materials.

In this specification, by composite it is meant a composition consisting of at least two different materials. The different materials may be for example ceramics, polymers or metals, such as hydroxyapatite, bioactive glasses or bioactive agents. These materials may also be in various forms such as particles, fibres or mats. A mixture according to the invention may be for example a mixture of two or more different sols or a sol and a gel. It is also possible to form a mixture of for example a gel and different particles, whereby, after treatment according to the invention, said particles are attached to each other by means of the treated gel.

According to one embodiment a sol is prepared from organic precursors through different chemical reactions so that a stable suspension is obtained. This sol is then transformed into a gel through further chemical reactions, such as polymerisation or aggregation, or through a “forced” gel forming. In a “forced” gel forming a substrate, such as implant, is e.g. dipped into or otherwise coated with a sol, the sol is transferred to the surface of the substrate, and when the liquid phase of the sol is evaporated the resulting gel is formed to the substrate surface. The gel, whichever way obtained, can then be treated according to the present invention.

In all the methods according to this invention, the energy may be selected from energies that can be localised, such as different laser irradiations, infrared irradiation, ultraviolet irradiation, visible light, X-ray irradiation, microwave irradiation, ultrasound waves, radioactivity, electron beam irradiation, acoustic waves, pressure waves and particle beam irradiation. It is also possible to use a combination of two or more energies. It is obvious that the choice of energy is determined by the biologically active sols, gels, mixtures or composites of sols and gels, and/or sol-gel derived materials used, the sensitivity of the additive or substrate to the energy or by the catalyst. For example, for silica or titania gels ultraviolet or infrared light, among others, are suitable.

One has to bear in mind that in traditional sintering in an oven, irradiations are also present. Said irradiations are however not localised as in the invention.

The energy is chosen preferably so that it specifically affects the network former molecules and causes them to form oxygen bridges by condensation reaction, alter their structure into denser or more solid form (densification and consolidation) or to at least partially sinter the gelated ceramic materials.

The energy is preferably used as impulses, whose frequency, intensity and advancement speed may be varied according to the result to be obtained.

The advantage of using localised energy source is that the desired treatment can be stimulated locally. In some cases, this means that lower vacuums may be used than with the prior art techniques. Another advantage of the invention is that by using filters or scanning of the energy, a defined pattern can be made on the device to be treated or on a coating. Yet another advantage of the invention is that the heat expansion coefficients of the used materials are not critical.

The advantage of treating materials with light or another low-thermal energy source is thus to avoid raising the temperature and therefore avoid destroying organic or other temperature sensitive components. The dissolution rate of the material is also fairly easily controllable.

According to one embodiment of the present invention it is possible to treat sols, gels, mixtures or composites of sols and gels, and/or sol-gel derived material locally with electromagnetic and/or acoustic energy. This enables the patterning, i.e. different parts or areas of the treated material can be treated in different ways. For example, one part of the material can be treated according to the invention and the second part of the material can be left untreated. For example, an implant, which comes into a contact with both soft and hard tissue when implanted into a body, can be partially treated according to the invention so that the first part of the implant which comes in contact with hard tissue is treated and the second part of the implant which comes into contact with soft tissue is left untreated. This way the hydroxyapatite formation is enhanced on that part of the implant, which comes into a contact with hard tissue, and a better bonding between the hard tissue and implant can be obtained.

Further advantages of the invention is that medical implants can be coated with a dissolvable ceramic coating including coatings containing a biologically active agent, and that sol-gel produced fibres can be strengthened while spinning.

According to an embodiment of the invention, the biologically active sols, gels mixtures or composites of sols and gels, and/or sol-gel derived materials comprise or act as a biologically active agent. The term “biologically active agent” shall be understood as an agent causing a valuable effect in vivo, such as a bioactive effect (i.e. promoting the binding of a tissue to an artificial implant inserted into the mammal body or other systems containing living organism), a therapeutic effect or the like. The term covers also agents useful for attaching bioaffinity groups such as antibodies, antigens, nucleotides etc. to a surface of a device for use in a bioaffinity assay. Such “attaching groups” are for example members of an affinity pair such as biotin-streptavidin or the like.

The invention also relates to a method for coating a device with biologically active sols, gels, mixtures or composites of sols and gels, and/or sol-gel derived materials. Said method is characterised in that at least one layer of biologically active sols, gels, mixtures or composites of sols and gels, and/or sol-gel derived materials is deposited on the surface of the device and that each said layer is sintered with a localised electromagnetic and/or acoustic energy prior to the eventual deposition of another layer of biologically active sols, gels, mixtures or composites of sols and gels, and/or sol-gel derived materials. It is obvious that it is also possible to use two or more different energies for a same process, for example in order to obtain areas of different structure and biological activity. A person skilled in the art also understands that it is possible to use the inventive method for partially coating a device, for example for filling grooves or holes on the surface of a device.

The device may be any device known per se, such as an implant, a fibre or a bone nail and it may be made of any desired material, for example metal (titania, platinum or gold), natural (mammal or vegetal) or artificial material such as cellulose or polyethene. The device may also have been previously treated, for example it may have been coated with a similar material as used in the invention, with the difference that this first coating has been sintered in an oven. In general, any device or surface may be treated with the inventive method. According to an embodiment of the invention, the device is manufactured from a biocompatible material and is for example a delivery device, such as an implant. The thickness of each layer of biologically active sols, gels, mixtures or composites of sols and gels, and/or sol-gel derived materials deposited on the surface of the device may be from 10 nm to 1000 nm, preferably from 10 nm to 200 nm, more preferably from 40 nm to 80 nm.

The layer of biologically active sols, gels, mixtures or composites of sols and gels, and/or sol-gel derived materials is deposited on the surface of the device by any method known per se, such as dipping, spinning or spraying. Said methods may also be used in any other application explained in this specification. Another useful method is chemical treatment in which the surface to be covered by sols, gels, mixtures or composites of sols and gels, and/or sol-gel derived materials is etched.

The invention still relates to a method for modifying the biological activity of biologically active sols, gels, mixtures or composites of sols and gels, and/or sol-gel derived materials by treating at least a portion of the surface with a localised electromagnetic and/or acoustic energy. It is also possible to completely destroy the biological activity of a certain area of the biologically active sols, gels, mixtures or composites of sols and gels, and/or sol-gel derived materials and to increase or decrease the activity of other areas. The destruction of activity is usually performed by a very strong energy: The modification of the biological activity depends on the amount of the energy used.

It is also possible to modify the activities of a pre-produced device or to form a device that already has the desired activities on desired areas by treating it according to the invention.

The invention further relates the use of the inventive method for the manufacture of devices consisting essentially of biologically active sols, gels, mixtures or composites of sols and gels, and/or sol-gel derived materials.

Different devices may be prepared by the method of the invention, such as fibres, monoliths, granulates, woven and nonwoven mats, tissue-guiding devices as well as films. The preparation of these devices is further discussed below. By tissue-guiding device it is meant a device that has such properties that once in place in the patient's body, it guides the formation of different types of tissues on different portions of the device. It may for example be a reinforcing mat whose first side is biologically active and thus promotes the formation of new tissue and the second side is biologically inert. It may also be a device of a desired shape having various channels through its body in order to guide the formation of a vein in these locations.

One further use of the inventive method is tape casting, wherein an emulsion containing particles is casted as a tape and said tape is then treated according to the invention. The treatment may be performed on desired portions of the tape.

A yet another aspect of the invention is the attaching of different devices. Said attaching is characterised in that the joint between said at least two devices is formed by at least partially treating the biologically active sols, gels, mixtures or composites of sols and gels, and/or sol-gel derived materials present in the location of the joint according to the inventive method. According to an embodiment of the invention, at least one of the devices has been coated according to the method described above. One or several of the devices may also have been manufactured from biologically active sols, gels, mixtures or composites of sols and gels, and/or sol-gel derived materials, or from any other material.

Preparation of Sols, Gels, Mixtures or Composites of Sols and Gels, and/or Sol-Gel Derived Materials

The controllably dissolvable silica-xerogels can be prepared by allowing silica-alkoxide, such as tetraethylorthosilicate (TEOS), to react with water at low temperature such as from −20° C. to +100° C., preferably at room temperature, in the presence of an acidic, e.g. acetic acid, or a basic catalyst by hydrolysis and polycondensation. Optionally an additive, e.g. ethanol or polyethylene glycol, or a combination of additives may be used. Preferably, the catalyst is acidic. The biologically active agent can be added to the reaction mixture at any stage of the process. However, it is preferable to add the biologically active agent to the reaction mixture at the sol-stage before polycondensation reaction takes place or mix it with the starting materials. The gel is formed into the desired form, and water, other liquids and optional additive(s) are removed from the gel by washing and drying to produce the silica-xerogel, which can be further partially sintered, consolidated or densified.

Release of the Biologically Active Agent

The silica gels dissolve controllably, and the release of the biologically active agent from the device is based on dissolution and/or slow diffusion from the pores (titania gels, for examples), which allows constant local release of the biologically active agent into the tissue. The release rate of the biologically active agent can be controlled via factors such as the pore structure of the material, the elemental composition of the gel and the dimensions of the gel.

Biologically Active Agents

The biologically active agent can be any organic or inorganic agent that is biologically active. The biologically active agent can be, e.g. a medicine, a protein, a hormone, a living cell, a dead cell, a bacteria, a virus or a part thereof. Biologically active agents include those especially useful for long-term therapy, such as hormonal treatment, e.g. contraception and hormone replacement therapy and for the treatment of osteoporosis, cancer, epilepsy, Parkinson's disease, pain and cognitive dysfunction. The suitable biologically active agents may be, e.g. anti-inflammatory agents, anti-infectives (e.g. antibiotics and antiviral agents, such as glindamycin or miconazole), analgesics and analgesic combinations, antiasthmatic agents, anticonvulsants (e.g. oxycarbazepine), antidepressants, antidiabetic agents, antineoplastics, anticancer agents (e.g. toremifene, tamoxifene, taxol), antipsychotics, antispasmodics, anticholinergics, sympatomimetics, cardiovascular preparations, antiarrythmics, antihypertensives, diuretics, vasodilators, CNS (central nervous system) drugs such as antiparkinsonism drugs (e.g. selegiline), steroidal hormones (e.g. estradiol, progesterone, nestorone), sedatives (e.g. atipamezole, dexmedetomidine, levomedetomidine), tranquillisers and cognitive dysfunction drugs. The medicine can be in the form of a salt, such as selegiline hydrochloride, (−)-4-(5-fluoro-2,3-dihydro-1H-inden-2-yl)-1H-imidazole hydrochloride, 4-(5-fluoro-2,3-dihydro-1H-inden-2-yl)-1H-imidazole hydrochloride, dexmedetomidine hydrochloride and toremifene citrate. The medicine can also be in the form of a free acid, such as ibuprofen; a free base, such as caffeine or miconatzole; or a neutral compound, such as Z-2-(4-(4-chloro-1,2-diphenyl-but-1-enyl)phenoxy)ethanol. A peptide can be e.g. levodopa and a protein can be e.g. an enamel matrix derivative or a bone morphogenetic protein. An effective amount of a biologically active agent is combined with sols, gels, mixtures or composites of sols and gels, and/or sol-gel derived materials. The precise amount employed in a particular situation is dependent upon numerous factors, such as the method of administration, type of mammal, the condition for which the biologically active agent is administered, the particular biologically active agent used, the desired duration of use etc.

Implants

It has also been found that the silica or titania gels can be used for implantable medical devices. A medical device can be implanted into any human or animal tissue. This allows local application so that targeting of the biologically active agent release site is possible. Therefore, the maximum effect from the agent is received. This part of the disclosure discloses different devices prepared or coated according to the inventive method. Said devices may be used in a method for administering a biologically active agent into a human or animal body, wherein said method comprises implanting, injecting or transmucosally attaching a delivery device or an implantable device.

For example, the silica-xerogels of the invention dissolve totally during the period desired when they are in contact with body fluids. Thus, delivery devices and medical devices prepared from these silica xerogels dissolve totally and controllably.

A delivery device may be for example a sol, gel, mixtures or composites of sols and gels, and/or sol-gel derived materials with a biologically active agent incorporated into the structure. A pharmaceutical preparation, such as a granulate or a capsule, in this context, is a preparation that comprises the delivery device and possibly additional excipients useful in implants and transmucosal preparations. A medical device may also be useful for orthopaedic and surgical purposes, e.g. for its bioactive effect, and need not to contain a biologically active agent incorporated into the structure of the sol, gel, mixtures or composites of sols and gels, and/or sol-gel derived materials. A medical device may be, e.g. a woven or nonwoven mat made of fibres.

The devices of the invention may be in various forms, e.g., in the form of a particle, a disc, a film, a membrane, a tube, a hollow particle, a coating, a sphere, a semi sphere, or a monolith, and they have various applications. It has been found that the form of the device can also control the dissolution rate of the device.

The biologically active agent in the material is released as the material dissolves and/or slowly diffuses from the pores. The release rate can be controlled via factors such as the pore structure of the material and the elemental composition of the material.

Particles of gel may be produced in different ways. The traditional gel forming-drying and then crushing results in particles that dissolve at the same rate as the bulk material per unit surface area. Crushing may slightly increase the surface area of micro- and mesoporous gels. Thus, the dissolution rate of the material per unit weight may increase accordingly. Monodispersed particles of silica are produced with an alkali gelation process. The sol gelates in spherical particles because of the alkaline conditions and, by preventing the final coagulation of the particles, separate monodispersed gel particles can be achieved.

Spheres and semi spheres are produced by spray drying or aerosol method. The sol described above is used for spraying. The aerosol droplets can be sprayed into gaseous or liquid suspension. During spraying into air, the small droplets dry in the atmosphere sufficiently to result in gelation of the hydrolysed ions. If the droplets hit a surface before sufficient drying, they will from semi spheres caused by surface energy differences between the droplet and the substrate. In that case, they will also gelate as semi spheres. If the sol is sprayed into a liquid suspension, they will gelate in suspension through the normal polymerisation process that takes place in the sol. The gelated particles can be sieved out of the solvent and dried in air. The gelated particles are dried or aged at room or moderate temperature (usually under 100° C., preferably under 50° C.) that results in further polymerisation of the OH-groups. The heat or ageing treatment slows the dissolution of the particles significantly. Ions, such as Na, K, P, Ca, Mg, Al, and B may be incorporated in the particles in order to produce dissolvable and/or bioactive bone bonding particles.

Spray drying of the gel particles and ageing them in a desiccator gives homogeneous, faultless particles with slow dissolution. Controllably dissolvable fibres can be produced by sol spinning and further treating with a localised electromagnetic and/or acoustic energy. The production temperature can be kept near room temperature. The fibres can be incorporated with ions, such as Na, K, P, Ca, Mg, Al, and B, in order to produce dissolvable and/or bioactive bone bonding fibres. The fibre production technique gives homogeneous and faultless materials.

A monolith can be prepared e.g. by casting the sol.

Woven or nonwoven mats prepared from fibres according the invention can be used to separate two or more types of tissues from each other. They can also be used as bone repair mats. It is advantageous if the tissue-guide is dissolvable so that it does not need to be removed by second operation.

The medical device materials available today are in the form of glass granules or as polymer films when the stiffness of the material is not satisfactory in order to use them as orthopaedic implants. The implants made of fibre mats according to the invention can be made flexible and dissolvable.

Polylactic acid, polyglycolic acid and polykaprolacton are degradable polymers used in medical devices which, however, need to be reinforced to achieve and maintain sufficient strength long enough while the degradation reduces the strength of the matrix. Controllably dissolvable gel fibres and particles of the invention are ideal for this purpose since they have the sufficient strength and a controllable dissolution rate. They may also be used for strengthening plastic packing materials that may be made of polylactic acid, starch or any other biodegradable polymer.

Tooth-implants, hip-implants, knee-implants, mini plates, external fixation pins, stents (e.g. for use in repair of blood vessels) or any other metallic, polymeric, ceramic or organic implants can be coated with a layer of sols, gels, mixtures or composites of sols and gels, and/or sol-gel derived materials according to the invention and a biologically active agent may be incorporated into this coating. The coating dissolves in the tissue and releases the active substance locally.

Sols, gels, mixtures or composites of sols and gels, and/or sol-gel derived materials treated according to the present invention can be used for coating of several different substrate materials, such as titanium, alloys of nickel and titanium (NiTi-alloys), other memory-shape metals, Al₂O₃ or other ceramic materials.

A bone-collecting filter is a device placed on a suction tube, which removes the debris and excess liquids from the operation site. When the surgeon is drilling, sawing, grinding or otherwise working on bony tissue the bone chips can be collected with the filter and placed back into the defect. So far, these filters are not dissolvable in the tissue. If these filters were made of fibres or particles according to the invention, they could be made dissolvable and loaded with a biologically active agent. Thus, the entire filter could be placed into the defect site with the bone chips.

The materials treated according to the invention can be used as cell growth substrates in the form of membranes, coatings, monolith, fibres, woven or nonwoven mats. Cell growth assisting substances are released from the substrate with the dissolving material.

Particles according to the invention can be administered as a spray into sinuidal or lung tissue where they will slowly dissolve and release biologically active agents. Similarly, small particles can be injected in a carrier fluid in the tissues.

Another use of the inventive technique is in local gelation of the sol. Three-dimensional objects can be generated by locally gelating the sol by a laser beam for example. A thin layer is gelated according to the invention and then another layer of sol is added to get a thin over-layer on top of the gelated material. Then the treatment is repeated. In literature this system has been described for polymers as SLA (stereolitography) and SGC (solid ground curing).

The sol and gel materials used in these techniques are preferably silica, titania, alumina, calcium phosphates or zirconia based or mixtures of these or other biocompatible oxides or inorganic materials. Additives to these sol and gel materials can be polymeric, metallic, ceramic, organic or inorganic compounds, bacteria, viruses or cells. Additives can function also as energy absorption centres to catalyse the gelation or sintering process.

### DESCRIPTION OF THE DRAWINGS

The invention is further illustrated in the following drawings that show some embodiments of the invention.

FIG. 1 illustrates the coating of a device according to a first embodiment of the invention.

FIG. 2 illustrates the coating of a device according to a second embodiment of the invention.

FIG. 3 illustrates the modification of the biological activity according to the invention.

FIG. 4 represents schematically a tooth implant positioned in a patient's mouth.

FIG. 5 illustrates the surface of a device which biological activity has been modified according to the invention.

FIG. 6 illustrates the attaching of three devices according to a third embodiment of the invention.

FIG. 7 illustrates the attaching of two devices according to a fourth embodiment of the invention.

FIGS. 8a-8d illustrate the experimental design used for the production of a monolith according to an embodiment of the invention.

FIG. 9 illustrates the experimental design used in Example 1.

FIG. 10 shows a scanning electron microscopy (SEM) image of the surface prepared in Example 1, after immersion in SBF.

FIG. 11 shows an energy-dispersive X-ray (EDX) analysis of the surface prepared in Example 1, after immersion in SBF.

FIG. 12 shows a back-scattering SEM image of the surface prepared in Example 1.

FIG. 13 shows the change in calcium concentration of the surfaces of Example 1.

FIG. 14 shows the change in phosphate concentration for different surfaces.

FIGS. 15a and 15b illustrate the thickness of the coating in Comparative example 3 and Example 2, respectively.

FIGS. 15c and 15d illustrate the porosity of the coating in Comparative example 3 and Example 2, respectively.

FIGS. 16a and 16b show the treatment with laser in Example 4.

FIGS. 17a and 17b show SEM images of the surface according to Comparative example 5 and Example 4, respectively, before the immersion into SBF.

FIGS. 18a and 18b are SEM images of the surface according to Comparative example 5 and Example 4, respectively, after immersion into SBF for two days.

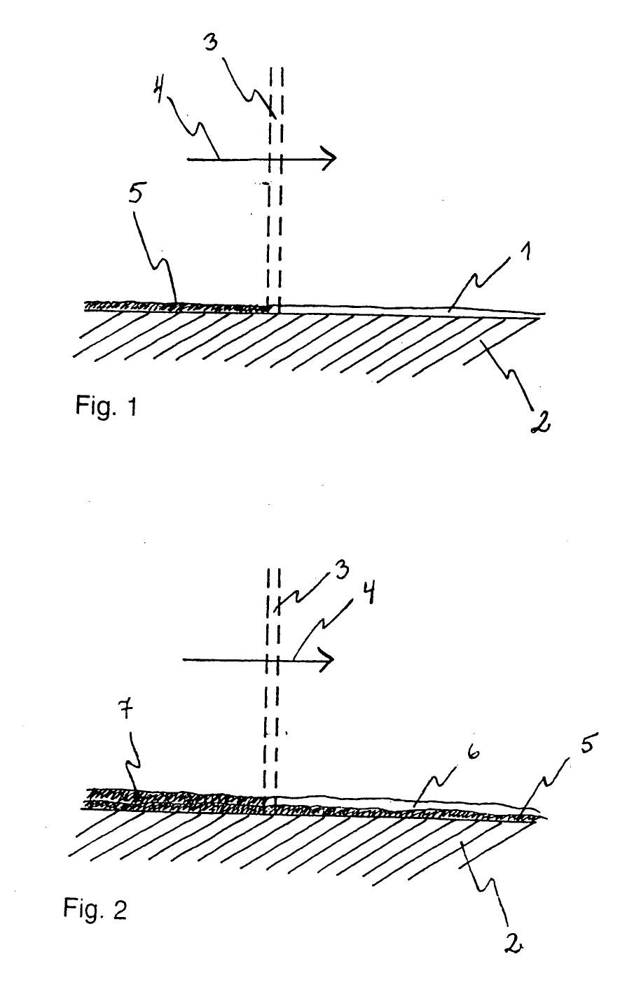

In FIG. 1, a layer 1 of titania-sol is deposited on the surface of a device 2. This layer is then scanned with CO₂-laser beam 3 in the direction indicated by the arrow 4 and under the effect of the laser beam, a treated coating 5 is formed on the surface of the device 2, according to this first embodiment of the invention.

In FIG. 2, a device 2 coated according to the embodiment shown in FIG. 1 is further coated with a second layer 6 of titania-sol. A second layer 6 of titania-sol is deposited on the first layer of coating 5 and the second layer 6 is then scanned with CO₂-laser beam 3 in the direction indicated by the arrow 4. Thus a thicker layer 7 of treated gel is obtained than in the first embodiment shown in FIG. 1.

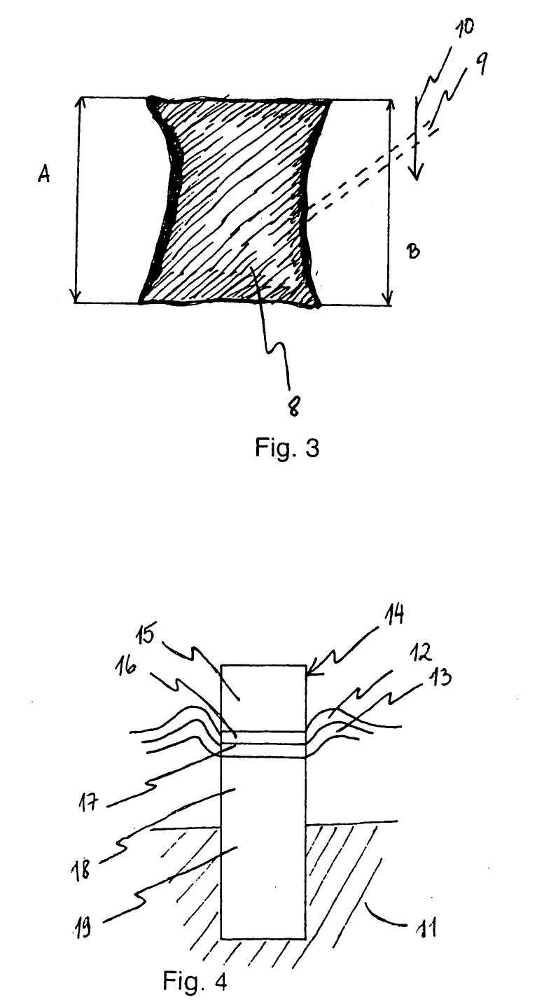

FIG. 3 shows the modification of the biological activity of a device made by the sol-gel technique. Desired portion of the surface of a device 8 that has been previously densified is treated with ultra-sound waves 9. In this embodiment, only the portions A and B of the surface are treated by scanning the waves 9 in the direction 10 on portion B and correspondingly on portion A of the surface. In this way, a different biological activity is obtained on portions A and B of the surface than on the other portions of the surface.

The implant 14 shown schematically in FIG. 4 is implanted in the jawbone 11. Two distinct layers 12 and 13 of gingival are also schematically represented. According to an embodiment of the invention, different parts of the surface of the implant are coated differently or the bioactivity of the coating is different in different parts of the implant. In this case, the coating of part 15 of the implant, that is the visible part once the implant is in place, has been treated with localised electromagnetic and/or acoustic energy so as to make it biologically inert. It is also obvious that part 15 may have no coating at all. The bioactivity of the coating of part 16, that is the part in contact with the first layer 12 of gingival is very low whereas the bioactivity of the coating of part 17, in contact with the second layer 13 of the gingival, is higher than the bioactivity of the coating of part 16.

The bioactivity of the coating of part 19 of the implant, that is the part that is in contact with the jawbone, is very high. Indeed, this part of the implant needs to form a strong bond with the jawbone and therefore part 19 is treated in such a way as to have increased bioactivity. Such increased bioactivity may have been obtained for example by different coating material or by different treatment (different energy or intensity). The bioactivity of the coating of the intermediate part 18 of the implant is between those of the parts 17 and 19.

FIG. 5 illustrates the surface of a device 20 which biological activity has been modified according to the invention. Indeed, the biological activity of the area 21 is different from the biological activity of the area 22 and of that of the area 23. Such a device may also be a so-called tissue-guiding device wherein different tissues are formed on the areas 21, 22 and 23.

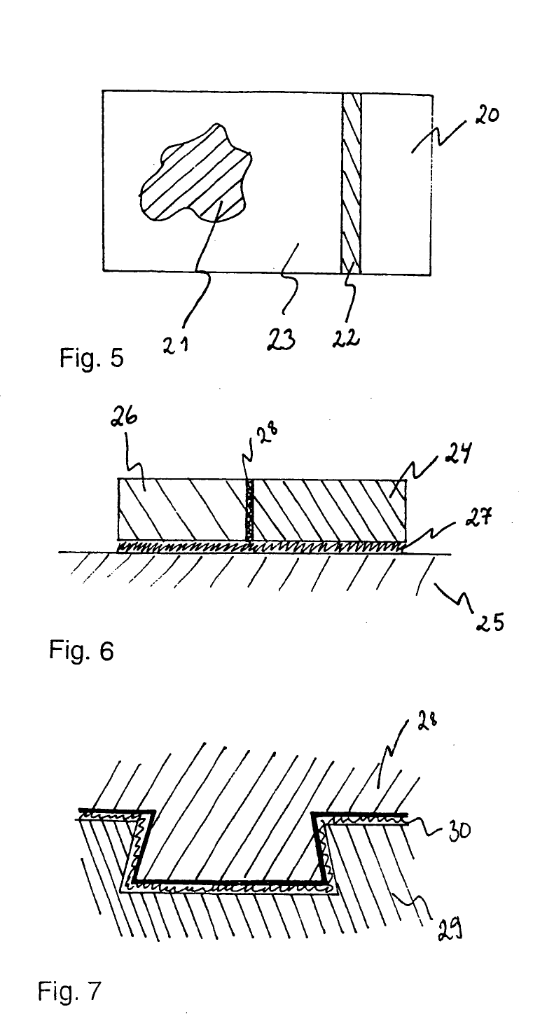

FIG. 6 illustrates the attaching of three devices 24, 25, 26 according to a third embodiment of the invention. Said three devices 24, 25, 26 may all be identical or different and they may be manufactured of any material. Said devices are attached by the means of biologically active sols, gels, mixtures or composites of sols and gels, and/or sol-gel derived materials 27, 28, the nature of which is not specified here. It is obvious to one skilled in the art that said biologically active sols, gels, mixtures or composites of sols and gels, and/or sol-gel derived materials 27 and 28 may be identical or different. It is however preferable if they are compatible, so that a strong bond is formed also at their intersection.

FIG. 7 illustrates the attaching of two devices according to a fourth embodiment of the invention. In this embodiment, the device 28 has been coated according to the inventive method and the device 29 has not been coated according to the inventive method (it may of course have been coated according to any other method). The devices are attached at the same manner than what has been explained above in relation with FIG. 6 and the joint is marked with reference numeral 30. It is obvious that device 29 might also be coated, or that only the device 29 might be coated and not the device 28.

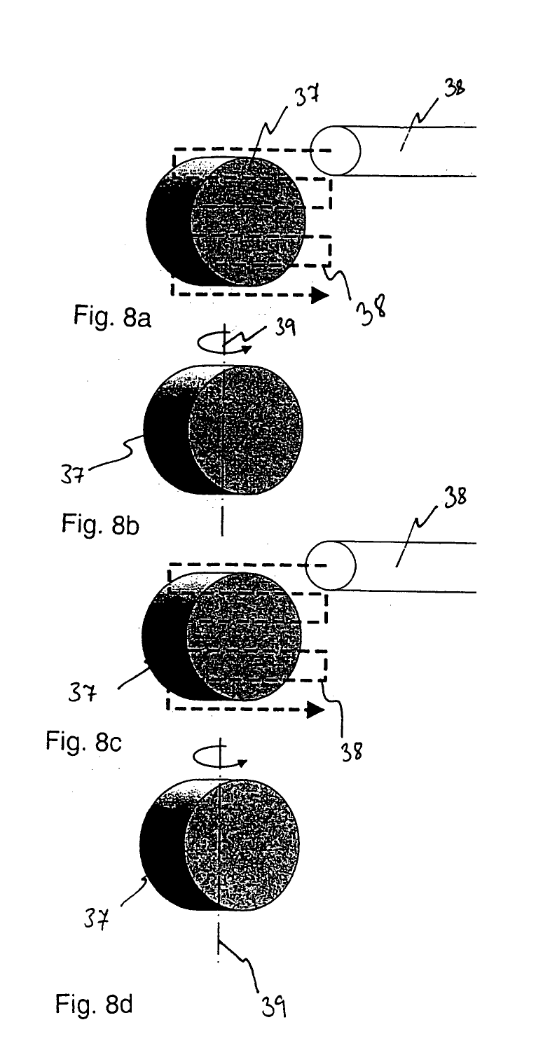

According to one of the embodiments of invention a monolith of silicon dioxide was manufactured. FIGS. 8a-8d illustrate the experimental design, which was used in manufacturing. A non-focused CO₂ laser beam 38 is scanned through a sol-gel derived SiO₂-monolith 37 following the pattern 38 (FIG. 8a). The monolith 37 is then turned 180° around its vertical axis 39 (FIG. 8b) and the scanning is repeated (FIG. 8c). The monolith 37 is again turned 180° around its vertical axis 39 (FIG. 8d) and the process can be repeated a number of times. It is obvious to one skilled in the art that the pattern 38 may be different than what is shown here and that it is possible to move the laser source instead of turning the monolith. It is also obvious that the manufacturing process herein described may be used for manufacturing any form of monolith.

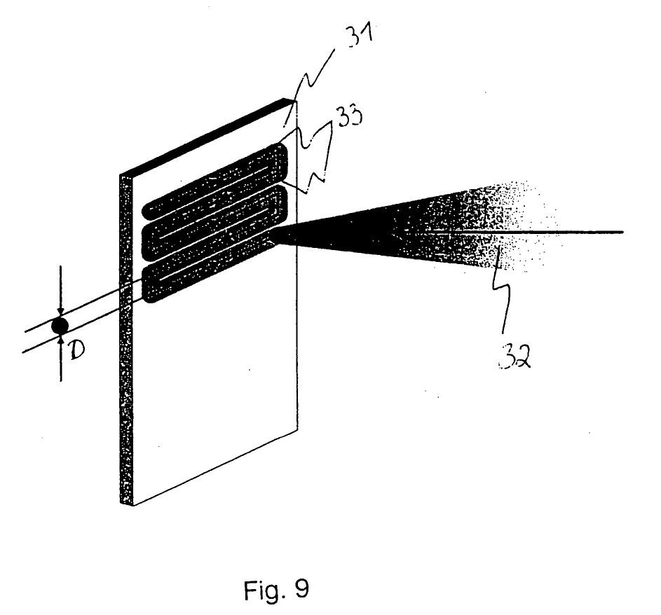

FIGS. 9-18 illustrate the Examples and are described below.

### EXPERIMENTAL SECTION

The following examples are intended to illustrate the invention, and are not to be construed as being limitations thereon.

### Example 1

The experimental design used for coating a substrate according to the invention is illustrated in FIG. 9. The substrate 31 is a titanium sheet and it was first dip-coated with a titanium dioxide (TiO₂) sol. A focused CO₂ laser beam 32 was then scanned through the substrate 31 in such a way that the distance D between two neighbouring scan lines 33 is equal to the diameter of the focused laser beam 32. The transition speed of the laser beam 32 was 2 mm/s and the power of the laser beam was 15 W. The substrate 31 was coated with five successive layers of TiO₂, that is, a first dip-coated layer was treated with laser beam, the substrate was further dip-coated and treated etc. It is obvious to one skilled in the art that the pattern may be different than what is shown here. It is also obvious that the coating process herein described may be used for coating any form and kind of substrate.

The final coated substrate was immersed in simulated body fluid (SBF) for 21 days. Simulated body fluid is a commonly used in vitro solution containing inorganic ions in concentrations corresponding to human blood plasma. The SBF used in these examples was prepared by dissolving reagent chemicals NaCl, NaHCO₃, KCl, K₂HPO₄.3H₂O, MgCl₂.6H₂O, CaCl₂.2H₂O and Na₂SO₄ into deionised water. The fluid was buffered at physiological pH of 7,40 at 37° C. with tris(hydroxymethyl)aminomethane and hydrochloric acid. It should be noted that SBF was used to simulate conditions in the body, it was not used as a coating fluid.

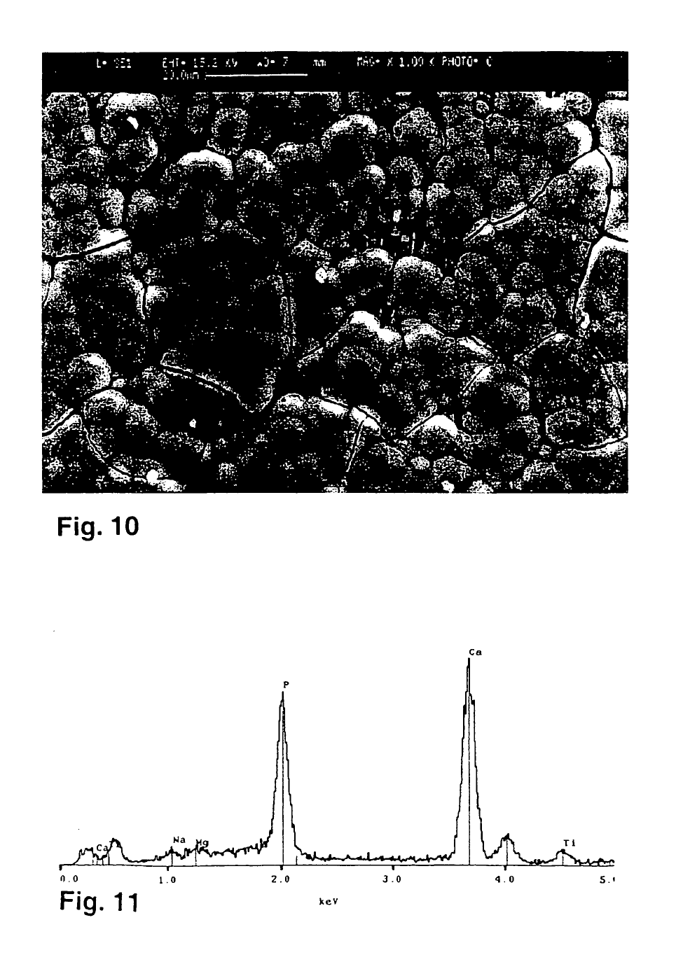

A scanning electron microscopy (SEM) image of the surface after immersion in SBF is shown in FIG. 10. The image shows mainly hydroxyapatite on titania gel. This hydroxyapatite is formed on the surface of the coated substrate first after immersion to the SBF. The surface, which has been treated according to the present invention, is therefore able to induce calcium and phosphate ions to the surface. Surface is then coated by the formed hydroxyapatite, and this coating occurs first when the treated surface comes into contact with SBF or real body fluids.

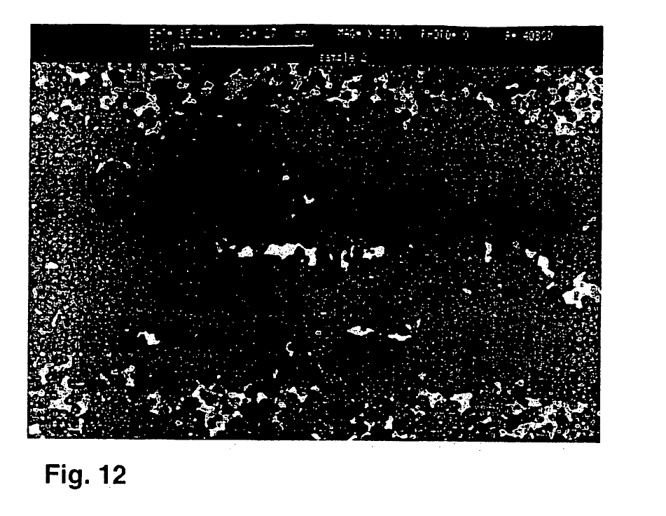

An energy-dispersive X-ray (EDX) analysis of the surface is shown in FIG. 11 and it reveals calcium (Ca) and phosphate (P) peaks, thus confirming that the surface has been covered with calcium phosphate after it has been immersed in SBF. Further, FIG. 12 shows a back-scattering SEM image of the coated substrate at ×250 magnification wherein the dark areas are covered with calcium phosphate and said dark areas follow the laser scanning pattern. This shows the formation of calcium phosphate to the treated surface after it came into contact with calcium and phosphate ions in SBF.

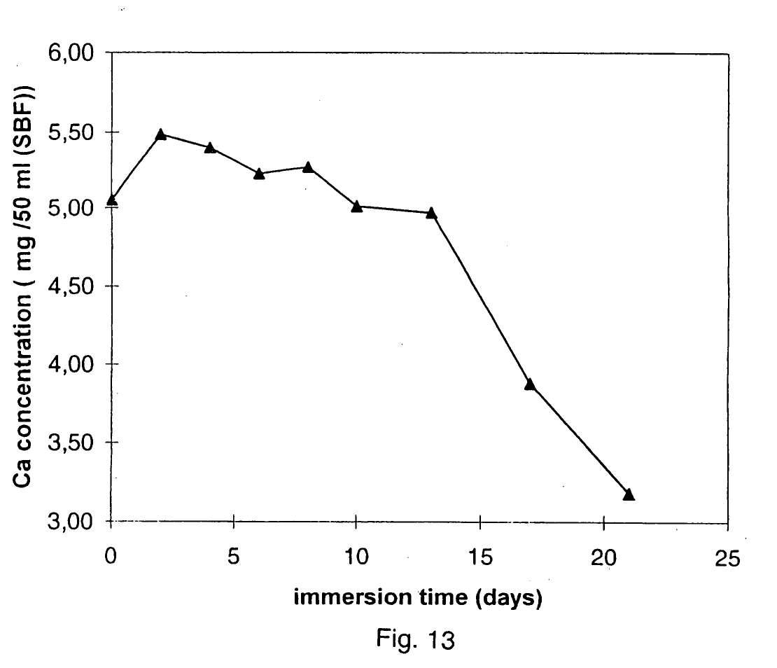

The bioactivity of the coating prepared in Example 1 was tested by immersing the coated substrate into simulated body fluid (SBF) and by comparing the concentrations of calcium and phosphate in the liquid. The concentrations are given as mg/50 ml SBF. FIG. 13 shows the change in calcium concentration of the SBF according to the immersion time.

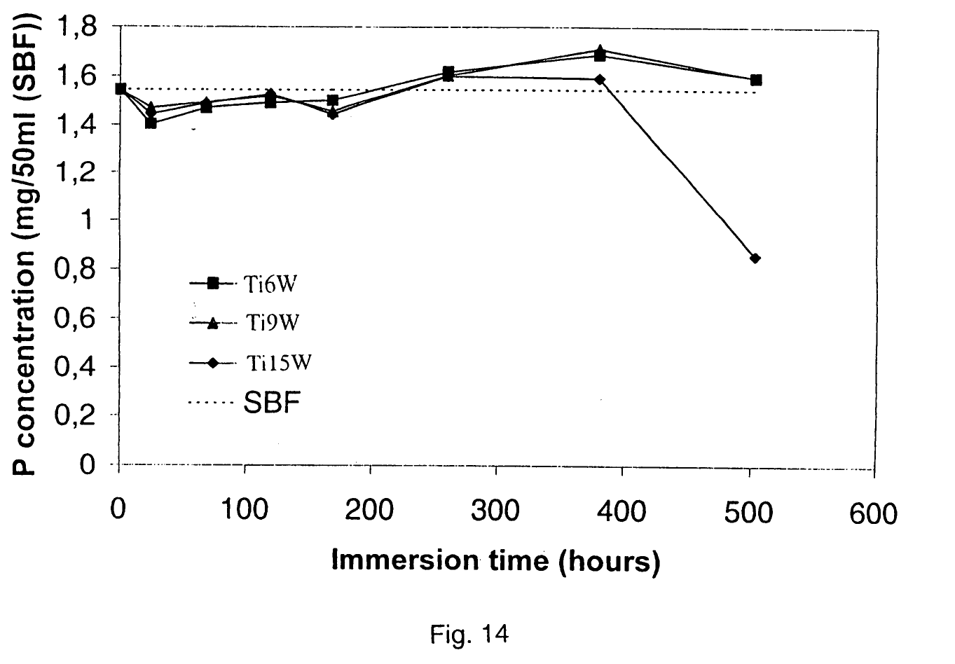

FIG. 14 shows the change in phosphate concentration of the SBF according to the immersion time and energies used. Three different laser powers were used, namely 6 W, 9 W and 15 W, the resulting samples being marked Ti6W, Ti9W and Ti15W, respectively. The sample marked SBF corresponds to a control sample of SBF.

As can be seen from FIGS. 13 and 14, the formation of calcium phosphate, which is similar to the bone mineral, is illustrated by the decrease of calcium and phosphate concentration in the immersion liquid. This evidence shows that the treatment according to the present invention enables the formation of calcium phosphate on the surface after it has been immersed into SBF.

### Example 2

A substrate of soda-lime-silicate glass (also known as microscope glass) was coated according to the invention by depositing a titanium dioxide sol and further treated with focused laser beam as in Example 1.

### Comparative Example 3

A substrate of soda-lime-silicate glass (also known as microscope glass) was coated by depositing a titanium dioxide sol and further sintered in an oven at different temperatures.

The surfaces prepared in Example 2 and in Comparative example 3 were then compared. The thickness of the coating and the porosity of the coating were evaluated by using UV-Vis spectral data and a computer program designed for this purpose and described in M. Ylilammi and T. Ranta-Aho, “Optical determination of the film thickness in multilayer thin film structures”, Thin Solid Films, 232, 56-62 (1993).

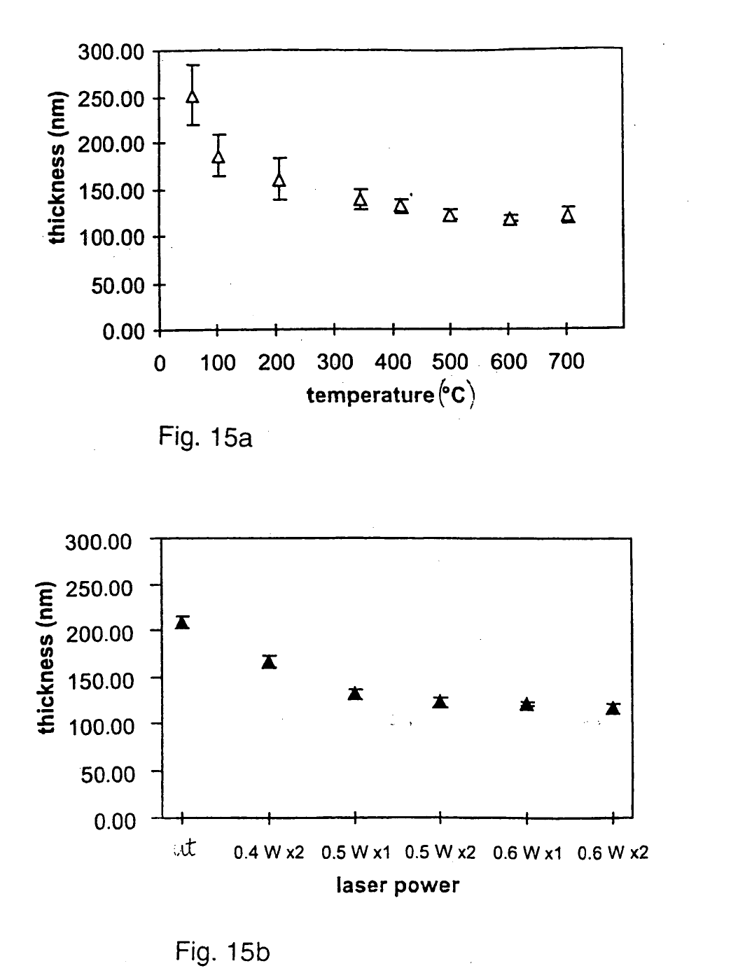

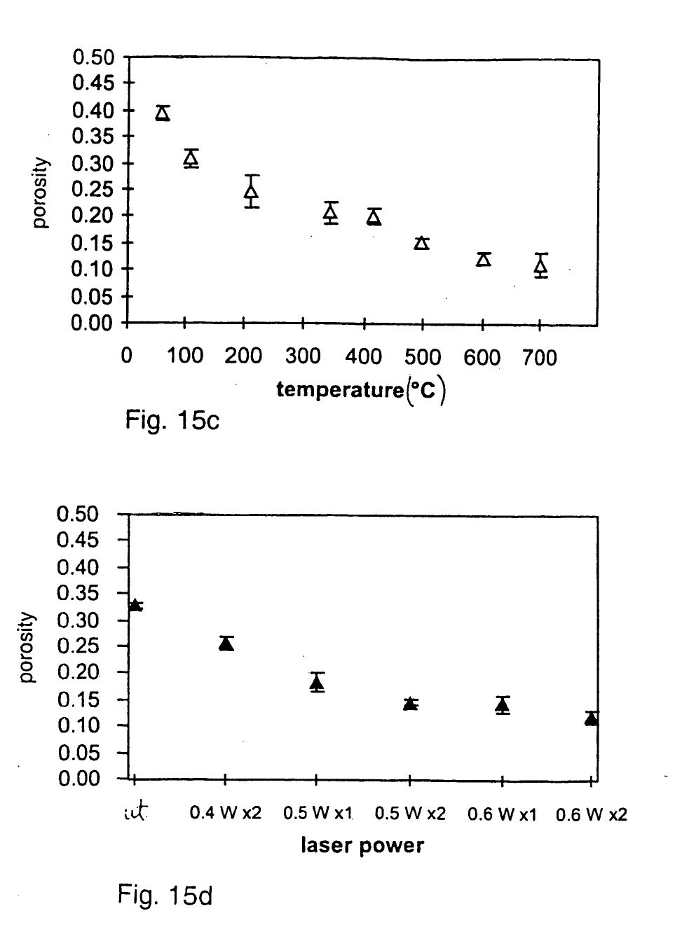

FIG. 15a illustrates the thickness of the coating according to the temperature, which coating has been prepared according to Comparative example 3. FIG. 15b illustrates the thickness of the coating according to the used laser power, which coating has been prepared according to Example 2. In FIG. 15b, “ut” means untreated. The reference marks used in the abscissa are as follows: 0.4 W×2 means that the surface has been treated twice with a power of 0.4 W, 0.5 W×1 means that the surface has been treated once with a power of 0.5 W, etc.

FIG. 15c illustrates the porosity according to the sintering temperature, which coating has been prepared according to Comparative example 3. FIG. 15d illustrates the porosity of the coating according to the used laser power, which coating has been prepared according to Example 2. In FIG. 15d, “ut” means untreated.

From these comparisons, it is obvious that the physical structure of the coating is essentially independent from the production method.

### Example 4

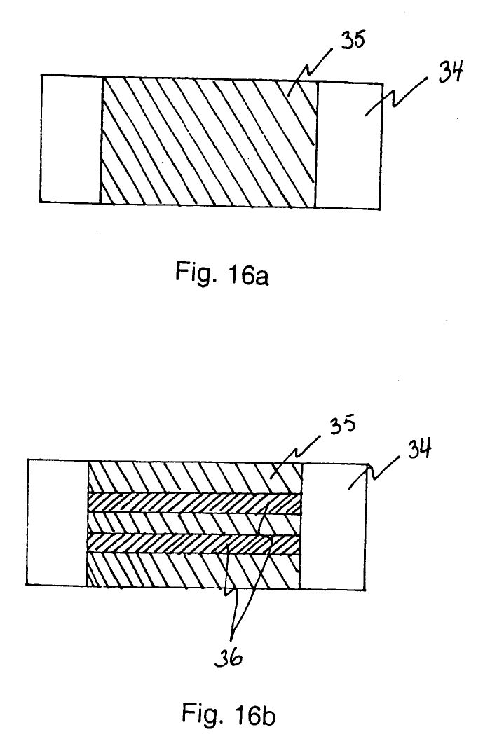

Titanium plates were dip-coated with a titanium dioxide sol. Each layer of coating was heat-treated at 500° C. for 10 minutes. The coating process was repeated four times, yielding to a coating of five layers. Restricted area of the surface was treated with focused laser beam according to the invention with the experimental design explained in Example 1 and shown in FIG. 9. Said treatment was performed immediately after the heat-treatment. The transition speed of the laser beam was 2,5 mm/s and the power of the laser beam was 20 W. FIG. 16a shows the coated surface 35 of the titanium plate 34 prior to the treatment with laser, and FIG. 16b shows the laser-treated areas 36. The width of the treated bands as well as their distance from each other was equal to the diameter of the focused laser beam.

### Comparative Example 5

Titanium plates were dip-coated with a sol-gel of titanium dioxide. Each layer of coating was sintered at 500° C. for 10 minutes in an oven and washed first with acetone, then with ethanol. The coating process was repeated four times, yielding to a coating of five layers.

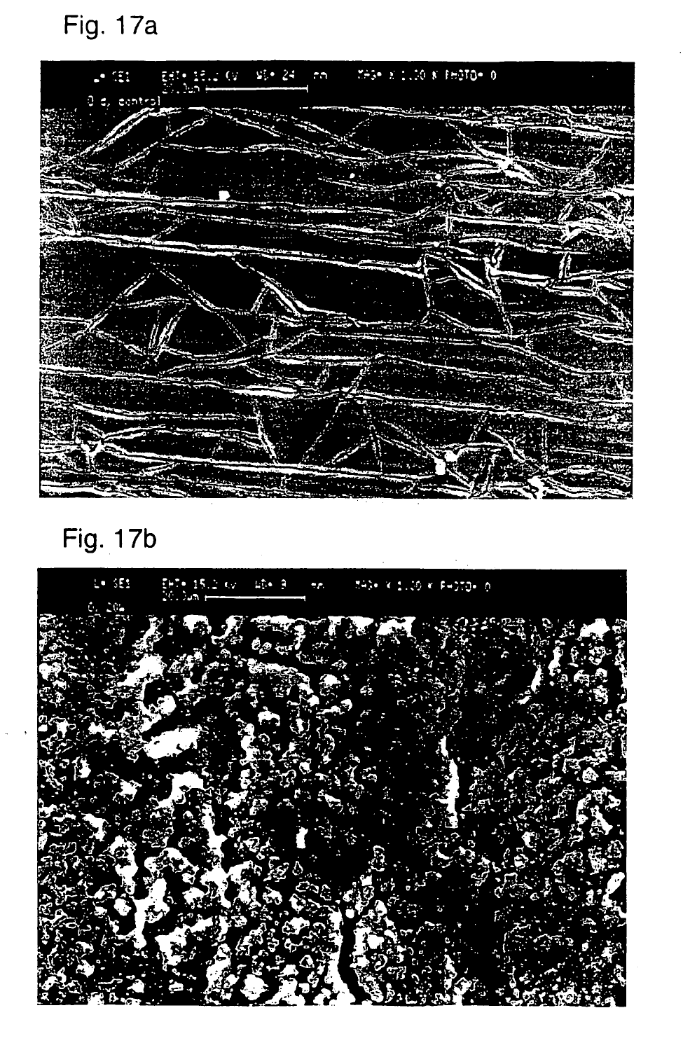

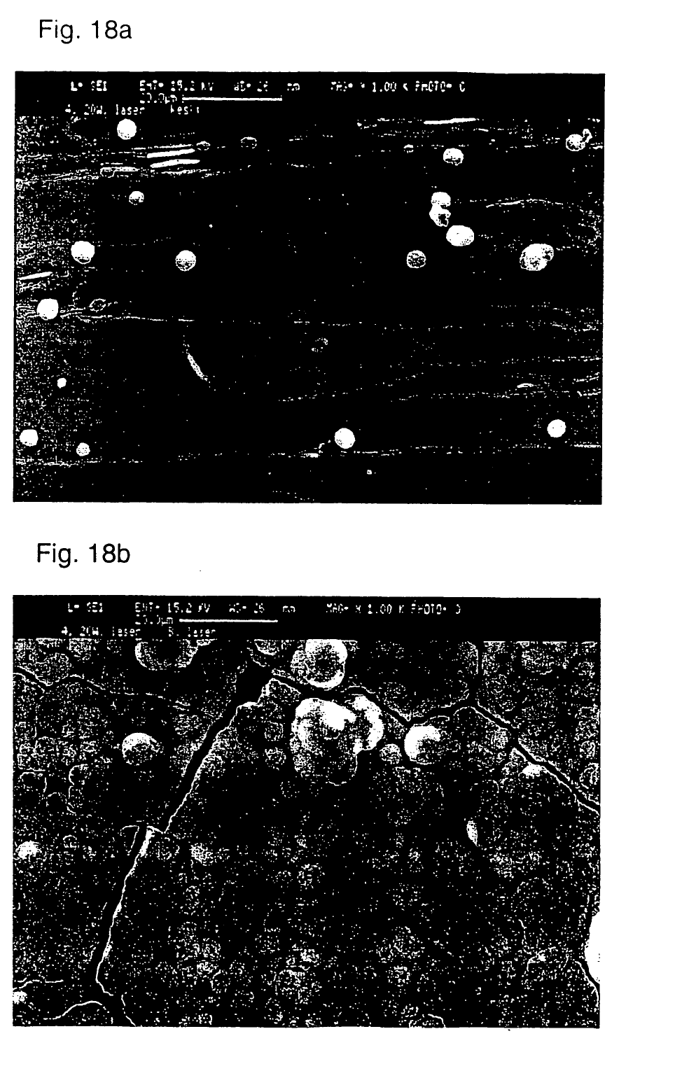

The coatings prepared in Example 4 and Comparative example 5 were compared by immersing the plates in to simulated body fluid as in Example 1. FIGS. 17a and 17b are SEM images of the surface prepared in Comparative example 5 and Example 4, respectively, before the immersion into SBF. FIGS. 18a and 18b are SEM images of the same substrate's surfaces after two days of immersion in SBF at room temperature. The spheres on the surface are calcium phosphate and one can see that the laser-treated area in FIG. 18b is covered with calcium phosphate whereas there is very little of it on the surface in FIG. 18a. This proves that the surface treated according to present invention effectively induces calcium and phosphate ions from the surrounding solution and enables the formation of calcium phosphate on the treated surface after the immersion to SBF.

1. Method for at least partially treating biologically active sols, gels, mixtures or composites of sols and gels, and/or sol-gel derived materials, said sols, gels, mixtures or composites of sols and gels, and/or sol-gel derived materials comprising OH-groups, being at least partially amorphous and selected from the group consisting of silica, titania, calcium phosphates, zirconia based and mixtures thereof, the said treatment being performed with a localised electromagnetic and/or acoustic energy.

2. Method according to claim 1, characterised in that said treatment consists of consolidation, densification, polycondensation and/or sintering.

3. Method according to claim 1 or 2, characterised in that the localised electromagnetic and/or acoustic energy is selected from the group consisting of laser irradiation, infrared irradiation, ultraviolet irradiation, visible light, X-ray irradiation, microwave irradiation and ultrasound waves.

4. Method according to any of claims 1-3, characterised in that the layer of biologically active sols, gels, mixtures or composites of sols and gels, and/or sol-gel derived materials comprises or acts as a biologically active agent.

5. A method for coating a device with biologically active sols, gels, mixtures or composites of sols and gels, and/or sol-gel derived materials, characterised in that at least one layer of biologically active sols, gels, mixtures or composites of sols and gels, and/or sol-gel derived materials, selected from the group consisting of silica, titania, alumina, calcium phosphates, zirconia based and mixtures thereof, is deposited on the surface of the device and that each said layer is treated with a localised electromagnetic and/or acoustic energy prior to the eventual deposition of another layer of biologically active sols, gels, mixtures or composites of sols and gels, and/or sol-gel derived materials.

6. Method according to claim 5, characterised in that the device is manufactured from a biocompatible material.

7. Method according to claim 5 or 6, characterised in that the thickness of each layer of biologically active sols, gels, mixtures or composites of sols and gels, and/or sol-gel derived materials deposited on the surface of the device is from 10 nm to 1000 nm, preferably from 10 nm to 200 nm, more preferably from 40 nm to 80 nm.

8. Method according to any of claims 5-7, characterised in that each layer of biologically active sols, gels, mixtures or composites of sols and gels, and/or sol-gel derived materials is deposited on the surface of the device by dipping, spinning, spraying or chemical treatment.

9. Method according to any of claims 5-8, characterised in that the layer of biologically active sols, gels, mixtures or composites of sols and gels, and/or sol-gel derived materials deposited on the surface of the device comprises or acts as a biologically active agent.

10. Method according to any of claims 5-9, characterised in that the device is a delivery device or a bioactive device.

11. Method according to any of claims 5-10, characterised in that the localised electromagnetic and/or acoustic energy is selected from the group consisting of laser irradiation, infrared irradiation, ultraviolet irradiation, visible light, X-ray irradiation, microwave irradiation and ultrasound waves.

12. A method for modifying the biological activity of biologically active sols, gels, mixtures or composites of sols and gels, and/or sol-gel derived materials, characterised in that at least portion of the surface of the biologically active sols, gels, mixtures or composites of sols and gels, and/or sol-gel derived materials, selected from the group consisting of silica, titania, alumina, calcium phosphates, zirconia based and mixtures thereof, is treated with a localised electromagnetic and/or acoustic energy.

13. Use of the method according to claim 1 for the manufacture of devices consisting essentially of biologically active sols, gels, mixtures or composites of sols and gels, mixtures or composites of sols and gels, and/or sol-gel derived materials.

14. A fibre, characterised in that it has been prepared according to the method of claim 1.

15. A monolith, characterised in that it has been prepared according to the method of claim 1.

16. A granulate, characterised in that it has been prepared according to the method of claim 1.

17. A woven or nonwoven mat, characterised in that it has been prepared according to the method of claim 1.

18. A tissue-guiding device, characterised in that it has been prepared according to the method of claim 1.

19. A film, characterised in that it has been prepared according to the method of claim 1.

20. A coating, characterised in that it has been prepared according to the method of claim 1.

21. A method for attaching at least two devices, characterised in that the joint between said at least two devices is formed by at least partially treating said biologically active sols, gels, mixtures or composites of sols and gels, and/or sol-gel derived materials present in the location of the joint according to the method of any of claims 1-4.

22. Method according to claim 21, characterised in that at least one of the devices has been coated according to any of claims 5-11.

23. Method according to claim 21 or 22, characterised in that at least one of the devices has been manufactured from biologically active sols, gels, mixtures or composites of sols and gels, and/or sol-gel derived materials.
